(19) United States
(12) Patent Application Publication
(10) Pub. No.: US 2026/0027557 A1
(43) Pub. Date: Jan. 29, 2026

(54) HIGHLY SELECTIVE CATALYST COMPOSITION FOR THE OXIDATION OF ALKENES TO EPOXIDES

(71) Applicants: The Regents of the University of California, Oakland, CA (US); THE ADMINISTRATORS OF THE TULANE EDUCATIONAL FUND, New Orleans, LA (US); TRUSTEES OF TUFTS COLLEGE, Medford, MA (US)

(72) Inventors: Phillip Christopher, Santa Barbara, CA (US); Anika Jalil, Santa Barbara, CA (US); E. Charles H. Sykes, Winchester, MA (US); Matthew Montemore, New Orleans,, LA (US); Laura Cramer, San Jose, CA (US)

(73) Assignees: The Regents of the University of California, Oakland, CA (US); THE ADMINISTRATORS OF THE TULANE EDUCATIONAL FUND, New Orleans, LA (US); TRUSTEES OF TUFTS COLLEGE, Medford, MA (US)

(21) Appl. No.: 18/998,095

(22) PCT Filed: Jun. 16, 2023
(86) PCT No.: PCT/US2023/068581

§ 371 Date: Jan. 23, 2025

Related U.S. Application Data
(60) Provisional application No. 63/392,370, filed on Jul. 26, 2022.

(51) Int. Cl. B01J 23/89; B01J 35/40; B01J 35/45; B01J 37/02; B01J 37/08; C07D 301/10

(52) U.S. Cl. CPC B01J 23/8946; B01J 23/892; B01J 35/40; B01J 35/45; B01J 37/0211; B01J 37/088; C07D 301/10

(57) ABSTRACT

A composition of matter useful for catalyzing an alkene epoxidation reaction using molecular oxygen (O₂) as an oxidant. including a plurality of structures comprising nanostructures or microstructures each comprising a coinage metal: and a plurality of single oxophilic metal atoms. The oxophilic metal is characterized by an oxide formation enthalpy being more exothermic than that of the coinage metal. In one or more examples. the oxophilic atom comprises nickel and the coinage metal comprises silver, and a concentration of the nickel increases selectivity to greater than 85% for the epoxidation reaction CH₂═CH₂+½O₂→(CH₂)₂O over combustion of ethylene forming carbon dioxide. and for an ethylene conversion of greater than 5%.

### CROSS REFERENCE TO RELATED APPLICATIONS

This application claims the benefit under 35 U.S.C. Section 119(e) of U.S. Provisional Application No. 63/392,370, filed Jul. 26, 2022, by Phillip Christopher; Anika Jalil; E. Charles H. Sykes; Matthew Montemore; Laura Cramer, entitled “HIGHLY SELECTIVE CATALYST COMPOSITION FOR THE OXIDATION OF ALKENES TO EPOXIDES,” which application is incorporated by reference herein.

### STATEMENT REGARDING FEDERALLY SPONSORED RESEARCH AND DEVELOPMENT

This invention was made with Government support under Grant (or Contract) Nos. DE-SC0021124 and DE-SC0004738, awarded by the Department of Energy. The Government has certain rights in this invention.

### BACKGROUND OF THE INVENTION

### 1. Field of the Invention

The present disclosure relates to catalysts useful for alkene epoxidation, methods of making the same, and systems performing the alkene epoxidation using the catalyst.

### 2. Description of the Related Art

Ethylene oxide is a high volume chemical used extensively in the chemical industry for the manufacturing of major chemicals and consumer products. In 2020, ethylene oxide had a global market of 29 million metric tons per year (about ˜40 billions USD) and the value of this market is expected to rise by ˜20 billion USD over the next 5 years. The catalytic oxidation of ethylene by molecular oxygen (O₂) is executed in industry using Ag-based catalysts supported on low surface area α-Al₂O₃. In patented technologies by the major EO manufacturers, Ag is usually promoted by other species such as Cs, Re, Li, etc. Further Cl is usually co-fed into reactors to improve performance. In reports on ethylene epoxidation in the academic literature and in patents, the best performance observed is about ˜90% selectivity to ethylene oxide (CO₂ is the other unwanted byproduct produced; selectivity is defined as the % of ethylene feed converted that produces ethylene oxide) and the per pass conversion of ethylene (% of ethylene fed to the reactor that has been converted to EO or CO₂ by the catalyst bed) is <15%. The relatively low conversion is uncommon in industrial chemical processes, as it then requires the reactants and products to be separated and a large recycle stream of the reactants to be fed back into the entrance of the reactor to create a continuous process. This separation and recycle process is extremely energy intensive, and thus intense research efforts over the past 50 years have been focused on continuing to improve the ethylene oxide selectivity and conversion. Because of the scale of this process, even a few % difference in EO selectivity between two different catalyst technologies represents a very significant difference in process economics (as characterized by differences in energy usage and associated CO₂ emissions). What is needed are methods that further increase the selectivity or conversion, or both, of alkene epoxidation processes. The present invention satisfies this need.

### SUMMARY OF THE INVENTION

The present disclosure reports on the surprising and unexpected discovery of a heterogeneous catalyst formulation that enables >85% (or even >90%) selectivity toward ethylene oxide formation from gas phase ethylene and oxygen, at high ethylene conversions of at least 5% (or even at least 11%). Embodiments of the discovery include the first instance of Ni doped Ag single atom alloy metal nanoparticles, which are supported on α-Al₂O₃, being used to catalyze ethylene epoxidation. Ni atoms dispersed in the Ag nanoparticles promote generation of selective oxygen species leading to the formation of ethylene oxide over the undesired product, carbon dioxide. The high selectivity retained at high conversion further suggests that the nature of the oxygen species on the NiAg catalyst mitigates the unwanted secondary reaction of EO combustion.

Illustrative embodiments of the inventive subject matter disclosed herein include, but are not limited to, the following.
1. A composition of matter, comprising:
   particles comprising a first component and a second component in a catalytically effective ratio wherein:
   the particles selectively catalyze a direct alkene epoxidation reaction using molecular oxygen (O₂) as an oxidant, when the particles are catalytically activated and used as a catalyst in the epoxidation reaction under reaction conditions, and
   the first component comprises at least one of silver, gold, or copper and the second component comprises at least one of nickel, indium, or gallium.
2. The composition of matter of example 1, wherein the first component comprises silver (Ag), the second component comprises nickel (Ni) and the catalytically effective atomic ratio Ni:Ag is in a range 1:100≤Ni:Ag≤1:1000.
3. The composition of matter of example 1, wherein the catalytically effective ratio is such that 10%-50% of the surface of each of the particles is composed of nickel under the reaction conditions.
4. The composition of matter of example 1, wherein the particles comprise less than 10 parts per million of caesium.
5. The composition of matter of example 1, wherein the catalytically effective ratio is such that the epoxidation reaction proceeds without a presence of chlorine.
6. The composition of matter of example 1, wherein the catalytically effective ratio increases selectivity to greater than 85% or greater than 83% for the direct epoxidation reaction CH₂═CH₂+½O₂→(CH₂)₂O over combustion of ethylene forming carbon dioxide, and wherein greater than 5% of the ethylene is converted to ethylene oxide.
7. The composition of matter of example 1, wherein the catalytically effective atomic ratio is 1:100≤Ni:Ag≤1:1000, the particles have an average diameter D
   50 nm≤D≤250 nm and 4*10\hat{ }-6≤Ni:Ag/D≤2*10\hat{ }-4 in units of nm\hat{ }-1.
8. The composition of matter of example 1, wherein a majority of the particles each have largest dimension D such that 1 nm≤D≤500 nm.
9. A catalyst for the epoxidation reaction comprising the composition of matter of example 1.
10. A composition of matter useful for catalyzing a direct alkene epoxidation reaction using molecular oxygen (O₂) as an oxidant, comprising:
   a plurality of structures comprising nanostructures or microstructures each comprising a coinage metal and a plurality of oxophilic atoms, wherein:
   a ratio of oxophilic metal: coinage metal is in a range 1:100≤oxophilic metal: coinage metal≤1:1000 and wherein a majority of the structures each have largest dimension D such that 1 nm≤D≤500 nm, and
   the oxophilic metal is characterized by an oxide formation enthalpy being more exothermic than that of the coinage metal.
11. The composition of matter of example 10, wherein the structures comprise less than 10 parts per million of caesium.
12. The composition of matter of example 10, wherein the structures comprise a nanostructured or micro-structured surface of a film or a porous structure.
13. The composition of matter of example 10, wherein the epoxidation reaction comprises ethylene epoxidation forming ethylene oxide.
14. The composition of matter of example 10, wherein a concentration of the oxophilic atoms comprising nickel in the coinage metal comprising silver increases selectivity to greater than 85% or greater than 83% for the direct epoxidation reaction CH₂═CH₂+½O₂→(CH₂)₂O over combustion of ethylene forming carbon dioxide, and wherein greater than 5% of the ethylene is converted to ethylene oxide.
15. The composition of matter of example 10, wherein the oxophilic metal is characterized by at least one of:
   an oxygen adsorption energy O_{A} for adsorbing oxygen on a crystal surface consisting of the oxophilic atoms, such that −5.8 eV≤O_{A}≤−5.4 eV as calculated using density functional theory (DFT) with the PW91 functional, a 396 eV cutoff, a 7×7×1 k-point grid for a 3×3×4 surface cell and according to
   the method and parameters in [19], and using gas-phase species of O as a reference state,
   a hydroxyl adsorption energy OH_{A} for adsorbing a hydroxyl group on the crystal surface, −3.12 eV≤OH_{A}≤−2.77 eV, as calculated using DFT with the PW91 functional, a 396 eV cutoff and a 7×7×1 k-point grid for a 3×3×4 surface cell according to the method and parameters in [19], and using gas-phase species of OH as a reference state, or
   an O2 dissociation barrier O_{B} for adsorbing oxygen on a single metal alloy of the oxophilic metal on the coinage metal consisting of Ag, such that 0.00 eV≤O_{B}≤0.25 eV, and a 2O adsorption energy 2O_{A} for adsorbing oxygen on the single metal alloy such that −2.62 eV≤2O_{A}≤−1.50 eV, as calculated using DFT with the PBE functional with TS correction, 400 eV cutoff and a 7×7×1 k-point grid for a 3×3×4

surface cell using the method and parameters in [20], and using adsorbed O₂ as the initial state for calculating O_{B} and gas-phase O₂ was as the reference state for calculating O_{A}.
16. A reactor for performing the epoxidation reaction, comprising an input for receiving the composition of matter of example 1 configured as a catalyst.
17. The reactor of example 16, wherein the reactor does not include a feed for feeding chlorine to the reaction.
18. A method of catalyzing an epoxidation reaction, comprising:
   contacting an epoxidation catalyst with an alkene and molecular oxygen, wherein the epoxidation catalyst comprises a coinage metal and an oxophilic metal, wherein the catalyst selectively catalyzes a reaction comprising a direct epoxidation of the alkene using the molecular oxygen (O₂); and
   outputting an alkene oxide formed by the reaction.
19. The method of example 18, wherein the coinage metal comprises silver and the oxophilic metal comprises nickel.
20. The method of example 18, further comprising pretreating the oxophilic metal in hydrogen (H₂) prior to catalyzing the reaction.
19. The method of example 18, further comprising performing the reaction in an absence of chlorine.
20. A method of making a composition of matter, comprising:
   forming structures each combining a coinage metal and an oxophilic metal in a catalytically effective ratio wherein the structures selectively catalyze a direct alkene epoxidation reaction using molecular oxygen (O₂) as an oxidant, when the structures are catalytically activated and used as a catalyst in the epoxidation reaction under reaction conditions.
21. The method of example 22, wherein the forming further comprises:
   providing a colloidal solution of the structures comprising nanostructures or microstructures comprising the coinage metal;
   combining a reducing agent and the oxophilic metal precursor with the colloidal solution; and
   performing a sequential reduction reaction wherein oxophilic metal is deposited from the precursor on the nanostructures or microstructures.

### BRIEF DESCRIPTION OF THE DRAWINGS

Referring now to the drawings in which like reference numbers represent corresponding parts throughout:

FIGS. 1A-1C. Schematic illustrations of example structures comprising a catalyst according to embodiments described herein, wherein FIG. 1A illustrates oxophilic atoms on particles on a substrate,

FIG. 2. (A) Density functional theory (DFT) calculations of the O₂ dissociation barrier with different metal dopants in Ag(111) and (B) temperature programmed desorption (TPD) of O₂ from on Ni_{1.5}Ag_{98.5} vs Ag(111) single crystal surface. (C) screening of various dopant elements within Ag for O₂ dissociation. (D) Table 1 of properties of various oxophilic metals.

FIG. 3. Secondary Ion Mass Spectrometry (SIMS) spatial mapping of different elements in colloidal NiAg₅₀₀ batch 1 solution. Images represent spatial maps in the x and y direction of (A) Ag (B) Ni (C) Na (D) Ag and Na (E) Ag and Ni (F) depth profiling of Ag and Ni signals

FIG. 4. Fourier Transform Infrared (FTIR) of NiAg₅₀₀ batch 1 after a 250° C. CO treatment. The spectra were taken after cooling the catalyst bed down to room temperature in CO, then purging with Ar. The 2055 cm⁻¹ feature is indicative of CO—Ni binding and the rest of the features are gas phase CO. As the cell is purged, gas phase CO leaves and only the bound CO is left behind (blue curve).

FIG. 5. Scanning Electron Microscopy (SEM) images and corresponding surface area weighted diameters of the NiAg₅₀₀ batch 1 catalyst after calcination and before reaction (A) and after reaction (B). The bright, white features indicate Ag nanoparticles while the gray features represent the α-Al₂O₃ support. The catalyst powder was directly drop casted onto carbon tape and topologically flat surfaces were accounted for the size distribution. The size distributions for the nanoparticles in (A) and (B) are shown in (C) and (D) respectively.

FIG. 6. Rates per gram of Ag of CO₂ formation (left) and ethylene oxide formation (right) as a function of the reactor temperature. Ag (black square), control Ag (gray square), NiAg₁₅₀ (green diamond), NiAg₄₀₀ (blue triangle), NiAg₅₀₀ batch 1 (red circle) and NiAg₅₀₀ (red square) are shown. Details of catalyst loading are in FIG. 6. Inlet feed was 10% O₂, 10% C₂H₄ balance He at 60 sccm for these measurements.

FIG. 7. Selectivity to ethylene oxide formation as a function of the ethylene conversion. Different conversions were achieved by varying the temperature of the reactor. Only data points with the legend starting with “Flow Rate Summary” indicate varying conversion by varying flow rate between 40-80 sccm. For NiAg₅₀₀ batch 2, flow rates of 40 sccm and 60 sccm were used to collect data.

FIG. 8. Stability test of 468 mg of 5 wt % NiAg₅₀₀ diluted in 2.4 g of SiO₂. The conditions for the test are: 40 sccm total flow rate, reactor temperature of 250° C. at atmospheric pressure and a 10% C₂H₄, 10% O₂ feed.

FIG. 9: Table 2. Summary of catalytic performances across different processes, where X denotes ethylene conversion and S denotes EO selectivity.

FIG. 10. Plot of fraction of Ni atoms versus nanoparticle size.

FIG. 11. Flowchart illustrating a method of making a catalyst according to one or more embodiments.

FIG. 12. Flowchart illustrating a method of using the catalyst.

FIG. 13. Schematic of a reactor for alkene epoxidation.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

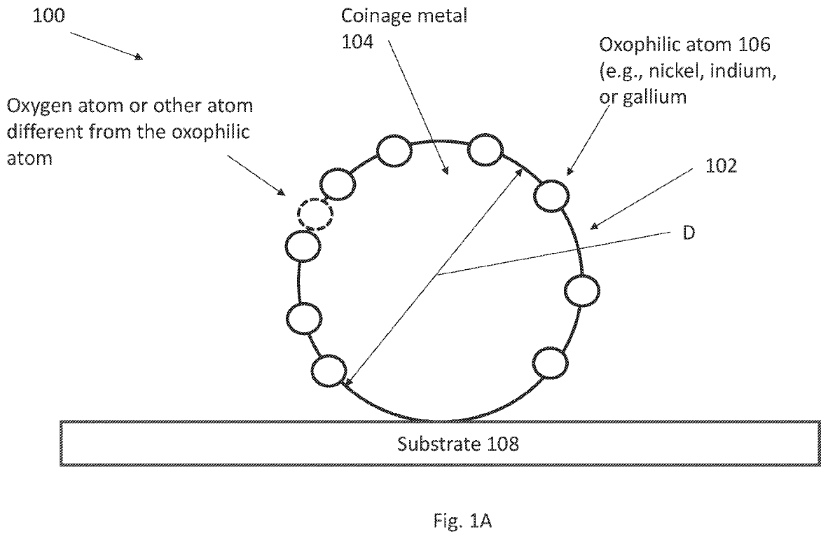

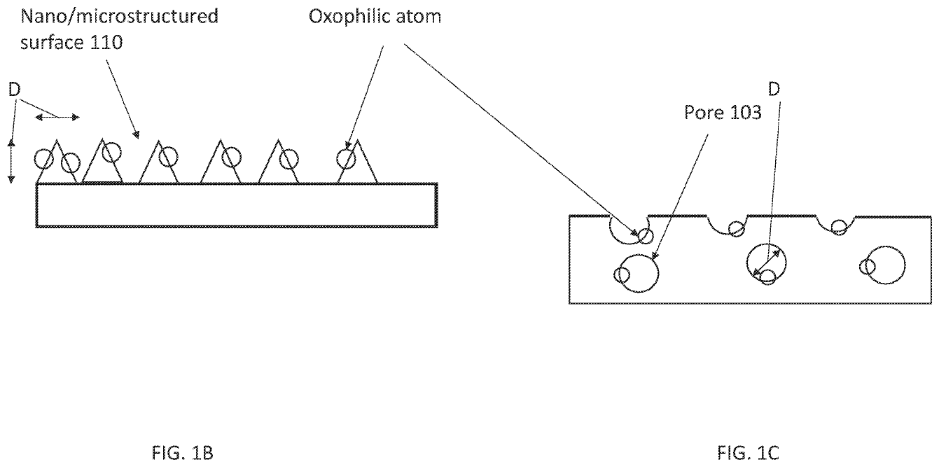

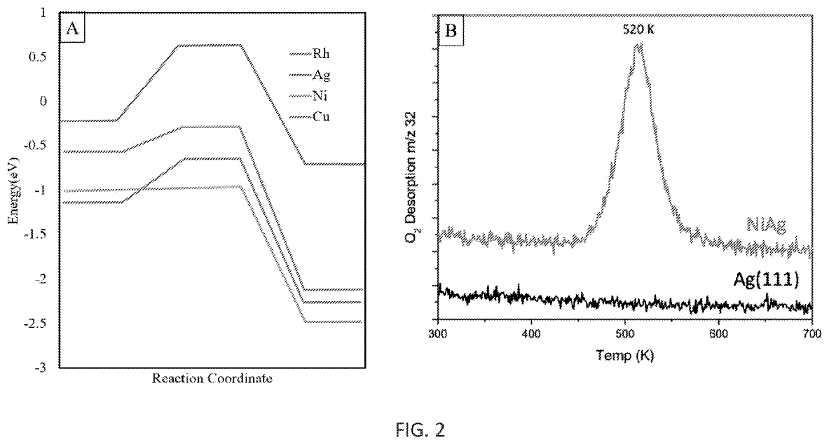

The present disclosure reports on a composition of matter useful for catalyzing an alkene epoxidation reaction using molecular oxygen (O₂) as the oxidant. FIGS. 1A-1C illustrate example compositions 100 comprising a plurality of structures 102 (e.g., particles as in FIG. 1A, surfaces as in FIG. 1B, or pores 103 as in FIG. 1C) comprising nanostructures or microstructures each comprising a coinage metal; and a plurality of oxophilic metal atoms combined with/attached to (e.g., in a surface of) the coinage metal. In one or more examples, the oxophilic atoms may be atomically dispersed on, and alloyed into a surface of each of the structures). In one or more examples, the oxophilic metal is characterized by an oxide formation enthalpy from the bulk metal (comprising the oxophilic metal atoms) being more exothermic than the coinage metal.

In various examples, each of the oxophilic atoms are attached or bonded to the coinage metal, e.g., so that each oxophilic atom forms a single atom alloy with the coinage metal in the structures. In one or more examples, each single atom alloy is defined as comprising a single atom of the oxophilic atom bonded or attached to the coinage metal (i.e., no bonds between the oxophilic atoms, e.g., no Ni—Ni bonds). FIGS. 1A-1C further illustrate each of the single oxophilic metal atoms is spaced from a next nearest one of the oxophilic atoms by at least one of an oxygen atom, a coinage metal atom, or another type of atom different from the oxophilic metal atom, thereby preventing bonding between the single oxophilic atoms and increasing a number of oxide bonds between the oxophilic atoms and oxygen from the molecular oxygen during the epoxidation. The oxophilic atoms may be on or partially in (e.g., partially embedded in) a surface of the structure.

In one or more examples, the oxophilic atoms comprise nickel, indium or gallium. However, other oxophilic atoms may be used as discussed herein.

The present disclosure further reports on the surprising discovery of catalytically effective atomic/stoichiometric ratios (of the oxophilic metal to the coinage metal) that enables greater than 83% selectivity for the direct epoxidation of alkenes. Example stochiometric ratios of the oxophilic metal atoms: coinage metal in each of the structures include, but are not limited to, a stochiometric ratio in a range of 1:3 to 1:10000, 1:3 to 1:1000, 1:10 to 1:1000, or 1:10 to 1:500.

In some examples, the surface concentrations of the oxophilic metal may be engineered to improve selectivity for the direct epoxidation reaction. Example surface concentrations of the oxophilic metal atoms (number of oxophilic metal atoms/total number of surface atoms)×100 include, but are not limited to, a surface concentration in a range of 0.001%-50%, 0.01% to 35%, 0.1% to 35%, or 1% to 35%, for example.

FIGS. 1A-1C illustrate the nanostructures or microstructures have at least one dimension D in a range of 1-10000 nm. In one or more examples, both the atomic/stoichiometric ratio and the micro/nanoscaled dimension D are simultaneously engineered to increase the selectivity for the direct epoxidation reaction. In one or more examples, the catalytically effective atomic ratio is 1:100≤Ni:Ag≤1:1000, the particles have an average diameter D 50 nm≤D≤250 nm, and 4*10\hat{ }-6≤Ni:Ag/D≤2*10\hat{ }-4 in units of nm\hat{ }-1.

The following examples illustrate various embodiments of the present invention.

### 1. First Example

### Density Functional Theory Calculations and Surface Science Experiments for Identifying Dopants

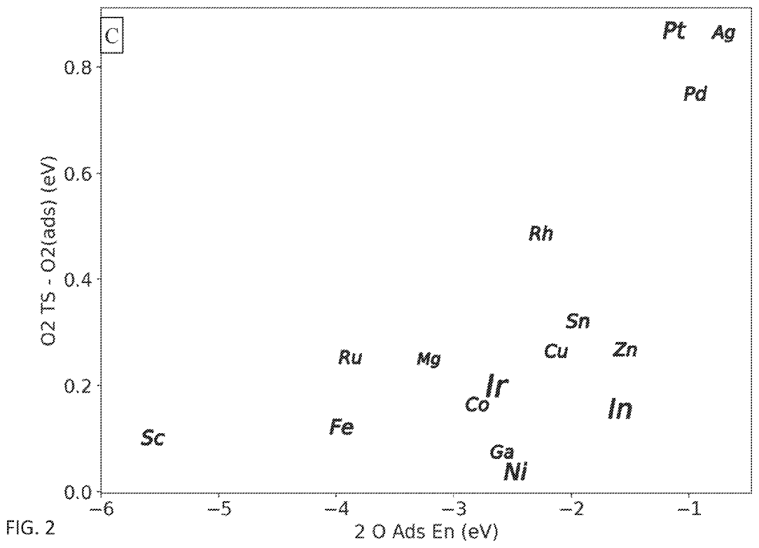

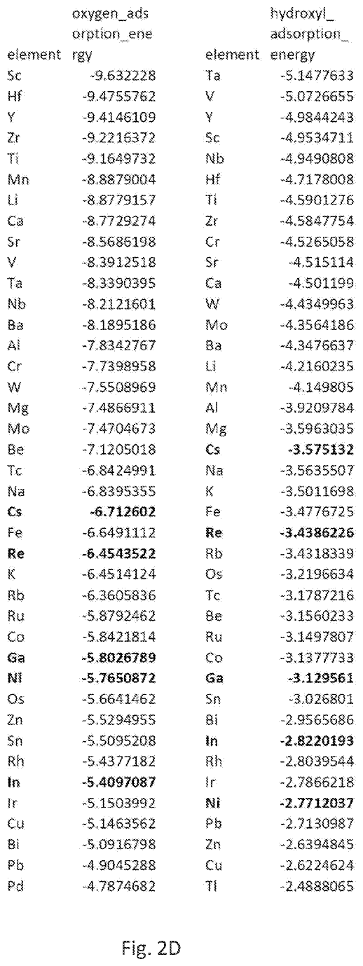

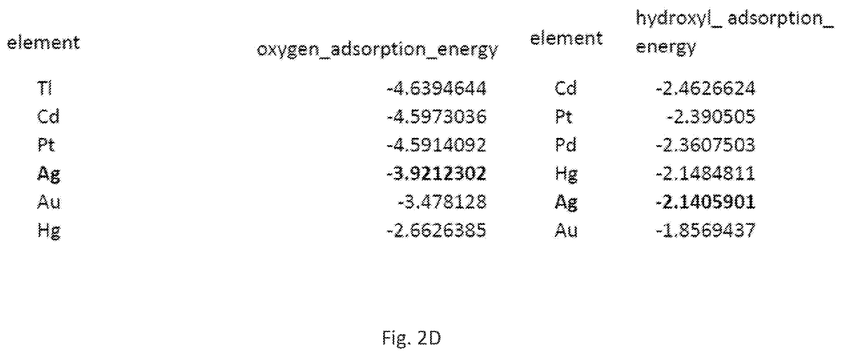

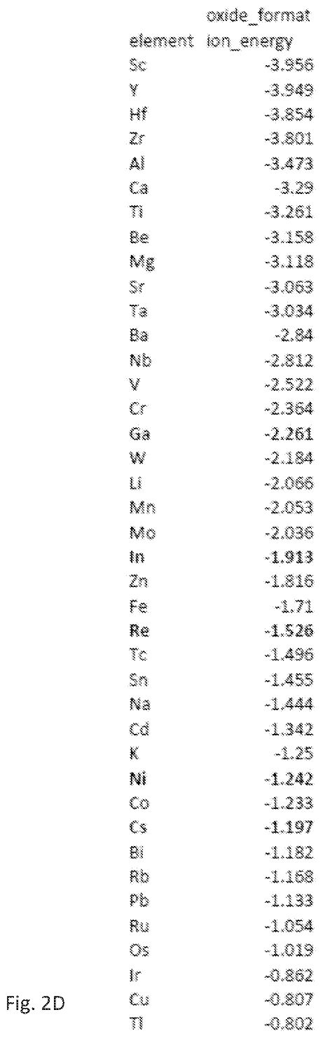

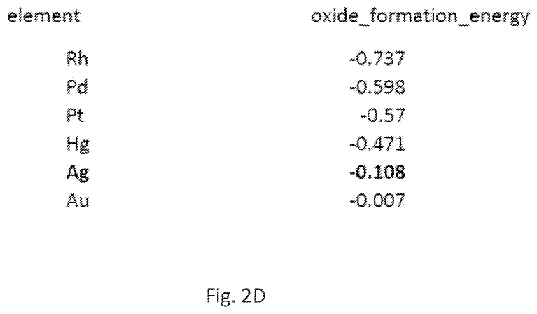

After screening various dopants within pure Ag, DFT calculations showed that Ni atoms doped into a Ag(111) surface as atomically dispersed species (single atom alloy, SAA) create active sites for nearly barrierless O₂ dissociation (see FIG. 2A and 2B). A TPD study of the NiAg single crystal surface at a Ni surface coverage of 1.5% (a coverage where Ni is dispersed as single atoms) showed that the NiAg surface has a significantly greater propensity to adsorb and dissociate O₂ compared to monometallic Ag when dosed with 5×10⁻⁴ Torr of O₂ at 350 Kelvin. Additionally, above 520 Kelvin, oxygen is shown to desorb molecularly from the surface indicating that the Ni—O bond in NiAg is not permanent. In ethylene epoxidation, the dissociative chemisorption of O₂ controls reaction rates while the population and nature of adsorbed O atoms on the Ag surface determines selectivity toward ethylene oxide formation.¹⁻⁴

The Table 2 in FIG. 2D tabulates various properties of oxophilic metals, as follows (blue highlighted entries are the most preferred dopants and red entries are example promoters).
oxygen_adsorption_energy: Calculated O adsorption energy on close-packed surface of native crystal structure (or similar if structure is complicated),
hydroxyl_adsorption_energy: Calculated top-site OH adsorption energy on fcc(111)
oxide_formation_energy: Experimentally measured oxide formation energy
M1Ag_O2_barrier: Calculated O₂ dissociation barrier for single-atom alloy of M in Ag(111). Initial state is adsorbed O₂.

M1Ag_2O_ads: Calculated 2O adsorption energy for SAA of M in Ag. Reference state is gas-phase O₂.
oxygen_adsorption_energy and hydroxyl_adsorption_energy were calculated with the VASP density functional theory program, using the PW91 functional, 396 eV cutoff, and a 7×7×1 k-point grid for a 3×3×4 surface cell. More details at source [19],.
M1Ag_O2_barrier, M1Ag_2O_ads were calculated with the VASP density functional theory program using the PBE functional with TS correction, 400 eV cutoff and a 7×7×1 k-point grid for a 3×3×4 surface cell. Same computational parameters as in [20].

In one or more further examples, oxophilic metals are characterized by at least one of (see FIG. 2D for definitions of various metrics that can be used to define the oxophilic atoms and methods used calculate the energies):
(1) an oxygen adsorption energy O_{A} for adsorbing oxygen on a crystal surface consisting of the oxophilic atoms, such that −5.8 eV≤O_{A}≤−5.4 eV, as calculated using density functional theory (DFT) with the PW91 functional, a 396 eV cutoff, a 7×7×1 k-point grid for a 3×3×4 surface cell and according to the method and parameter in [19], and using gas-phase species of O as a reference state;
(2) a hydroxyl adsorption energy OH_{A} for adsorbing a hydroxyl group on the crystal surface, −3.12 eV≤OH_{A}≤−2.77 eV, as calculated using DFT with the PW91 functional, a 396 eV cutoff and a 7×7×1 k-point grid for a 3×3×4 surface cell according to the method and parameters in [19], and using gas-phase species of OH as a reference state, or
(3) an O₂ dissociation barrier O_{B} for adsorbing oxygen on a single metal alloy of the oxophilic metal on the coinage metal consisting of Ag, such that 0.0 eV≤O_{B}≤0.25 eV, and a 2O adsorption energy 2O_{A} for adsorbing oxygen on the single metal alloy such that −2.62 eV≤2O_{A}≤−1.50 eV, as calculated using DFT with the PBE functional with TS correction, 400 eV cutoff and a 7×7×1 k-point grid for a 3×3×4 surface cell using the method and parameters in [20], and using adsorbed O₂ as the initial state for calculating O_{B} and gas-phase O₂ as the reference state for calculating O_{A}.

These findings from theory and surface science motivate the use of the Ni dopant (or other oxophilic dopants characterized by having an oxide formation enthalpy from the bulk metal being more exothermic than silver or the coinage metal host during epoxidation of the alkene) to modulate the nature of adsorbed O species on the Ag surface or coinage metal (including at the Ni or oxophilic site) and increase reaction rates or modify selectivity in ethylene epoxidation as compared to pure Ag, or both.

### 2. Second Example

### NiAg Alloys wherein the Oxyphilic Atom comprises Nickel and the Coinage Metal comprises Silver

A high surface area catalyst bearing the same active sites as proposed in the DFT and single crystal studies in the first example was examined.

a. Synthesis of High Surface Area NiAg₅₀₀ Single Atom Alloy (SAA) Catalyst

A NiAg SAA catalyst was prepared through a sequential reduction procedure adapted from various syntheses of bimetallic NiAu and NiAg materials in literature.⁵⁻⁷ A Ni:Ag molar ratio of 1:500 was selected to examine the situation where Ni is atomically dispersed as single atoms at the Ag surface, although other Ni:Ag molar ratios may also be used. The following method is for the synthesis of NiAg in the molar ratio 1Ni:500 Ag. Colloidal Ag nanoparticles were synthesized using the procedure outlined by Christopher et al. ⁸8 mL of ethylene glycol (VWR Chemicals) was heated with 1 g of PVP (Sigma-Aldrich, MW=55000-58000) to 165° C. in a glass vial with the cap screwed on. The PVP dissolved fully during the heating process to form a pale-yellow solution by gently agitating the vial during initial heating. Separately, 100 mg of AgNO₃ (Sigma-Aldrich) was dissolved in 3 mL of ethylene glycol by gently warming the solution on a heating plate with a setpoint of 50° C. for 1 minute. The AgNO₃ solution was quickly added to the PVP-ethylene glycol mixture and heated at 165° C. for 1 hour before cooling to room temperature. To the Ag colloid solution, 67 μL of a 5 mg/mL nickel nitrate hexahydrate (Sigma-Aldrich) solution dissolved in ethylene glycol was added followed by 20-40 μL of 50 wt % NaOH solution (Sigma-Aldrich) and 12.5 μL of hydrazine (35 wt % in H₂O, Sigma-Aldrich). The mixture was heated on a hotplate for 30 minutes with a setpoint of 150° C. but the temperature probe reading did not exceed 125° C. The cooled reaction mixture was then cleaned by centrifugation with ethanol until a viscous, ink-like liquid was left behind and the supernatant was a clear, pale-yellow. A fraction of the colloidal solution was then mixed thoroughly with α-Al₂O₃ (5-6 m²/g, Alfa Aesar, Lot: T12C026) and the liquid was evaporated such that the desired Ag loading of catalyst was obtained. The weight loadings of Ag and Ni are nominal loadings based on assumption of complete conversion of Ag precursor to final Ag structures and deposition onto the support. The rates per gram reported are conservative and are likely to increase upon ICP analysis. To synthesize pure Ag catalysts, the same procedure was followed with the omission of the second step. For the control Ag sample, the sequential reduction step is followed without the addition of the Ni precursor solution. After deposition of the colloids onto the support, the catalyst is calcined in air at 400° C. for 4 hours to remove organic molecules and other contaminants.

b. Characterization and Reactivity of the NiAg₅₀₀ Sample

### (i) SIMS

SIMS is a highly sensitive surface characterization technique, typically used in solid state chemistry as it enables the detection of elements down to the parts per billion range. An additional advantage is that SIMS imaging is performed on much larger length scales than energy dispersive X-ray spectroscopy (EDS), thereby providing a more statistically significant representation of the system.¹⁰ SIMS mapping of the elemental distribution of NiAg in the colloidal solution was performed with a Cameca IMS 7f Auto SIMS system. The sample was prepared by drop casting the colloidal NiAg solution onto a square of GaAs to improve sample conductivity. The elements were then mapped spatially in the x, y and z directions.

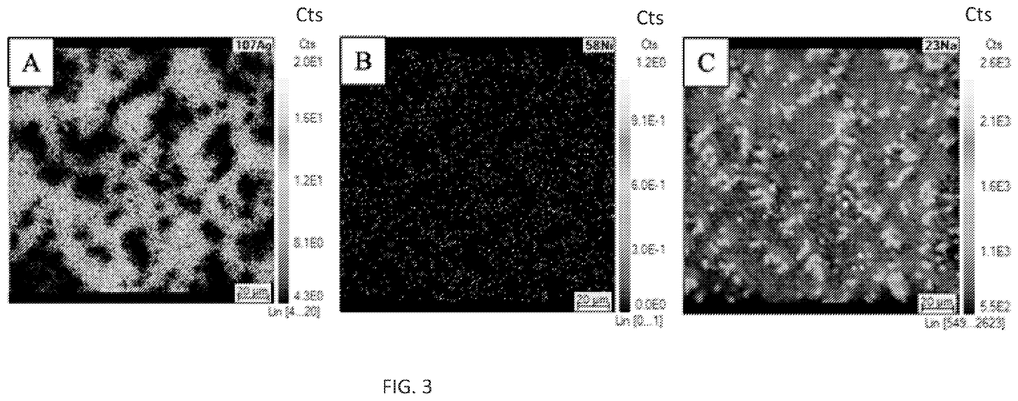

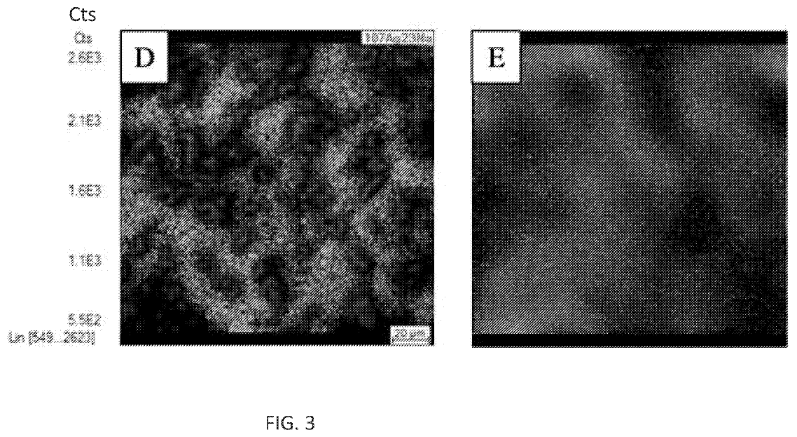

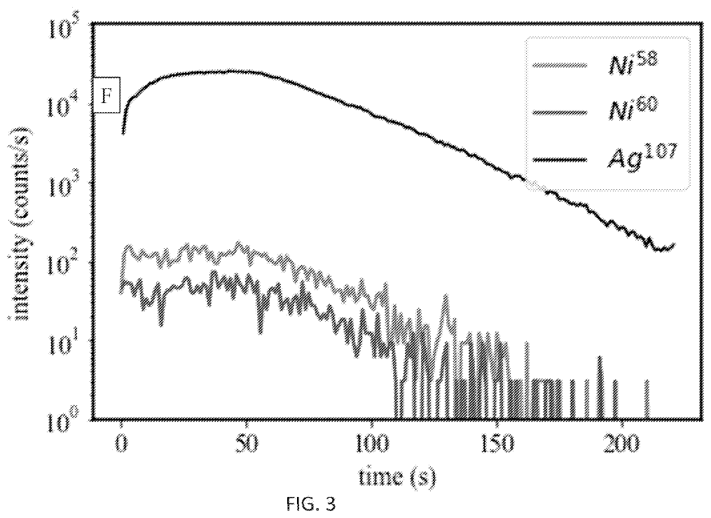

The SIMS spatial mapping (FIG. 3) showed a spatial correlation between Ni and Ag (FIG. 2) in the colloids. The signal on this measurement depends not only on concentration but also on the ionizability of the element. As expected, the dominant element in the colloidal solution is Ag owing to the high concentration of Ag present. Na is also seen in the measurement, but this signal is amplified by the affinity of Na to form Na⁺ ions rather than there being a high concentration of Na present. When the Ag and Na maps are overlayed on top of one another (FIG. 3D) there is little spatial correlation. When the Ni and Ag maps are overlayed, there is clear spatial correlation between the two elements. Depth profiling shows the intensity of Ag and Ni parallel to each other providing further evidence of physical association of the two elements. This is evidence that the synthetic approach was successful in incorporating Ni into the Ag sample.

### (ii) ICP-MS Measurement for Catalyst Composition

A small mass (<100 mg) of supported catalyst powder was dissolved in 0.1M nitric acid. A known volume of acid was added to the catalyst powder and either left to sit overnight or heated at 80° C. under reflux. The resulting solution was then separated from the support solids using centrifugation. The solution can then be diluted further to achieve an estimated Ni/Ag concentration assuming a nominal weight loading for the solids. A calibration curve was created on ICP-MS, using solutions of known concentrations as standards. Serial dilutions made from single or multi-use element ICP certified standards can be used for calibration. ICP-MS standards can be obtained from Inorganic Ventures or Sigma Aldrich. The composition of Ag and Ni can subsequently be determined using the following relationships (molar ratio equivalent to atomic ratio):

$$actual Ni or Ag wt \% = \frac{measured ppm}{calculated ppm} * expected Ni or Ag wt \%$$

$$actual \frac{Ni}{Ag} molar ratio = \frac{Ni wt \%}{{MW}_{Ni}} * \frac{{MW}_{Ag}}{Ag wt \%}$$

A method of using ICP-MS to determine the composition of an EO catalyst is described in [21]. Such a method can also be used to determine the atomic/molar ratio of oxophilic metal: coinage metal for embodiments described herein.

(iii) FTIR

CO-probe molecule Fourier transform infrared spectroscopy (CO-FTIR) experiments were performed by loading the catalyst into a Harrick Praying Mantis reaction chamber inside a Thermo Scientific Nicolet iS10 FTIR spectrometer. The catalyst was pretreated at 400° C. in H₂ to reduce the surface, cooled and then pretreated in 10% CO in Ar for 1 hour at 250° C. This technique allows the detection of surface Ni species in the Ag host as Ag does not adsorb CO. Thus, any signatures of adsorbed CO demonstrate that Ni is present at the Ag surface.

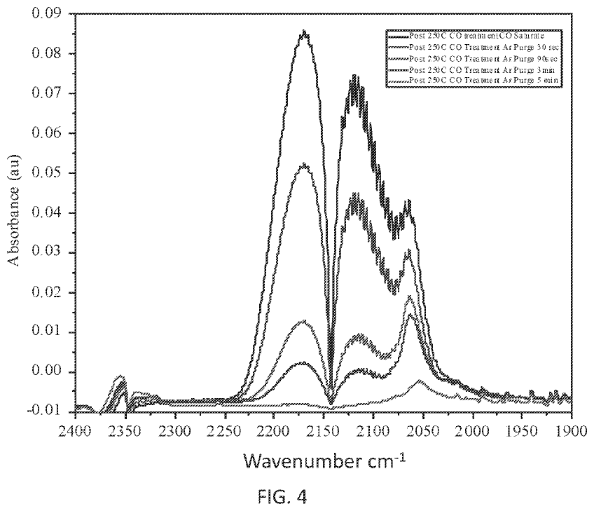

FIG. 4 plots the CO-FTIR spectrum characterizing the surface of the NiAg₅₀₀ catalyst after calcination and reduction in-situ. The data shows CO does not bind to Ag or the low surface area α-Al₂O₃ and pretreatment of the catalyst in CO brings Ni atoms from the bulk and pins them at the surface of the Ag particles (in line with theoretical projections that find that Ni prefers to segregate to the Ag surface in a single atom alloy configuration when CO is present).¹¹ The feature at 2055 cm⁻¹ is associated with on top adsorption of CO on Ni.¹² Studies of NiCu and NiAu single atom alloys also find a feature in the same location associated with CO—Ni binding.¹³ This is further evidence that Ni was incorporated in the Ag particles, and further that it is present at the Ag surface.

### (iv) SEM

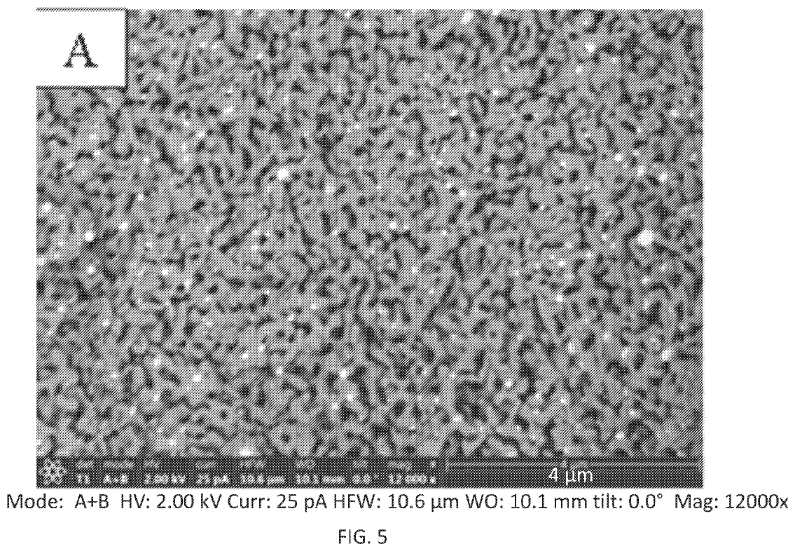

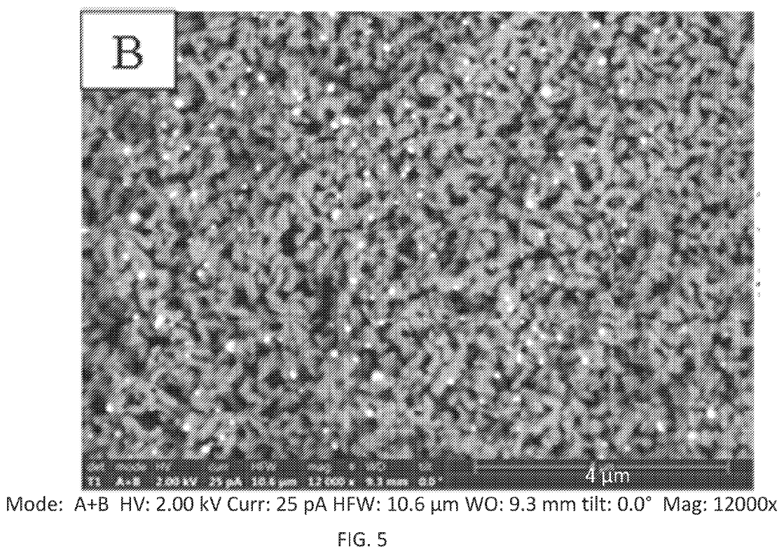

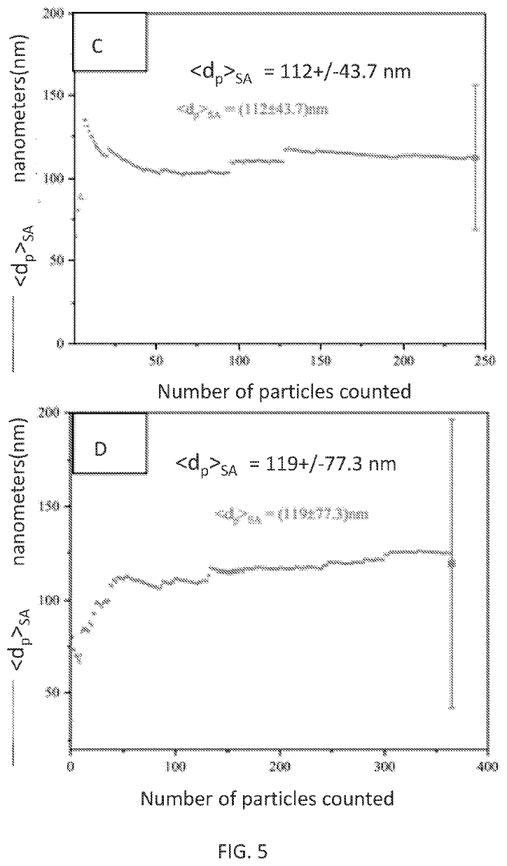

Scanning electron microscopy (SEM) images were taken of the unspent catalyst after calcination as well as the spent catalyst after reaction. ThermoFisher Apreo C LoVac FEG SEM was used with an accelerating voltage of 2.0 kV at a current of 25 pA to minimize artifacts from sample charging that can occur in a non-conductive support such as α-Al₂O₃. The SEM imaging showed no significant particle size growth between pre and post reaction samples (FIG. 5). Since surface coverage of Ni for a particular Ni:Ag ratio increases with particle diameter, this analysis supports that the surface concentration of Ni on the Ag particles remains relatively constant during the reaction. This would not be the case if significant sintering occurred, as this would change the Ag surface area. The synthetic method for large Ag particles (>100 nm diameter) allows for a rigorous calcination step by heating at 400° C. for 4 hours in air for full removal of PVP. Without being bound by a particular scientific theory, any particle growth likely occurred during this high temperature treatment, so there was little sintering at 250° C. since the system had already attained an equilibrium particle size under oxygen rich conditions at 400° C. This is consistent with a recent study by Egelske et al. that finds that Ag particles between 100-200 nm in size appear to exhibit high stability and a resistance to sintering.¹⁴

Measurements for particle size distributions in FIGS. 5C and 5D were made on the Apreo C SEM. The powdered samples are lightly sprinkled on conductive C tape on steel stubs and imaged using the z-contrast detector. The z-contrast detector picks up maximum contrast between the alumina support and heavier metallic nanoparticles for ease of analysis. For the size analysis, at least 200 nanoparticles were counted on image processing software (in this example: ImageJ). However, any suitable image processing software can be used (e.g., Python). The SEM image was first calibrated using the image scale bar to calculate length per pixel. The image was then converted to grayscale and thresholded to remove the pixel contributions from the support. Thresholding ensures that only Ag nanoparticles are counted. After image calibration and thresholding, rounded objects in an image were identified by adjusting circularity settings using either ImageJ or the Partycool package on Github. After taking these steps, calibrated areas for the particles were obtained. After quantifying area and corresponding particle diameters by assuming spherical nanoparticles, the mean particle diameter, surface area or volume weighted mean diameter and standard deviation of a population of nanoparticles were obtained. Assuming spherical particles, the following (for example) can be used to characterize the diameter:

$${d}_{p} = \frac{\sum {n}_{i} {d}_{i}}{N} ( arithmetic mean )$$

$$< {d}_{p} {>}_{SA} = \frac{\sum {n}_{i} {d}_{i}^{3}}{\sum {n}_{i} {d}_{i}^{2}} ( surface area weighted mean )$$

$$< {d}_{p} {>}_{V} = \frac{\sum {n}_{i} {d}_{i}^{4}}{\sum {n}_{i} {d}_{i}^{3}} ( volume weighted mean )$$

Where, ηᵢ is the count size in bin i, dᵢ is the diameter of the bin size and N is the number of particles counted. The data in FIGS. 5C and 5D plots the surface area weighted mean.

If the majority of particles are non-spherical in nature, the surface area of the high contrast Ag particles can still be thresholded to obtain the average particle surface area of the population by setting the lower bound of the circularity limit to a radius of zero.

### (iv) Catalytic Activity

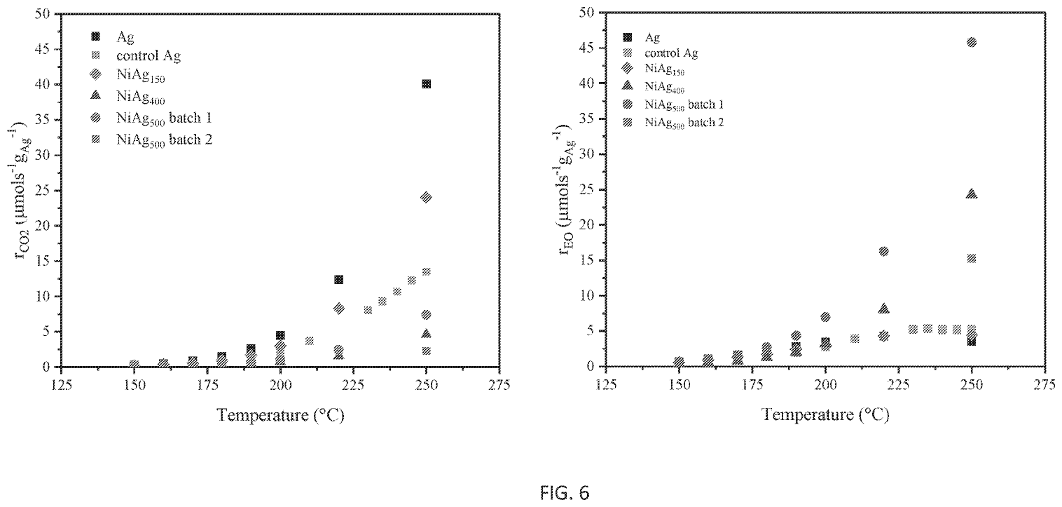

The catalytic activity of the catalysts was tested in a fixed-bed quartz reactor operating at atmospheric pressure and the results are shown in FIG. 6. Generally, 100-300 mg of the catalyst was vortexed with 5 times the amount of SiO₂ as dilutant and loaded in between two plugs of quartz wool. Gas flow rates were controlled using mass flow controllers from Teledyne Hastings. The reactor temperature was monitored using a thermocouple in the center of the quartz reactor and controlled using an Omega Engineering temperature controller. Experiments were performed between 150-250° C. at a total gas flow rate of 40-60 sccm. The inlet feed to the reactor was generally 10% O₂ (UHP, Airgas), 10% C₂H₄ (UHP, Airgas) and the inert He (UHP, Airgas). Ethylene conversion was varied by changing the temperature or inlet flow rate or mass of catalyst loaded. The reactor effluent was then analyzed using gas chromatography (GC, SRI 8610C) where O₂ and C₂H₄ partial pressures are monitored using the thermal conductivity detector (TCD) and CO₂ and EO formation is monitored using the flame ionization detector (FID). To calculate the FID sensitivity to EO, one data point was considered where 32.5 counts corresponded to an EO partial pressure of 2.87*10⁻⁴, although the data is consistent with EO production calculated based on C mass balance where the CO₂ and C₂H₄ calibrations are used to calculate the amount of EO required to close the mass balance (no other C based species were detected in GC). The reactor was allowed to sit at each temperature for 2-3 hours to ensure steady state measurement. Reported selectivities are defined as the moles of carbon in the desired product (EO) divided by the moles of ethylene consumed to make all the products (EO+½ CO₂ from the product perspective). The carbon mole balances (defined as difference in moles of carbon entering the system and moles of carbon exiting the system as a percentage of the moles of carbon that entered the stream) all close within 0.5-3.5%. EO selectivities of the pure Ag samples we made are consistent with values found in literature (40-50% at low ethylene conversion), validating the EO calibration estimate and general measurement approach.⁹ Before catalytic testing, the catalysts were pretreated in 10 sccm of H₂ (UHP, Airgas) at 400° C. for 1 hour and cooled in He before flowing reactant gases and ramping up to a temperature of 250° C. The system was allowed to stay at a steady state at 250° C., allowing it to “soak” for >12 hours before varying temperatures. This is so that any potential particle growth can occur at the highest temperature the system will be at and reach an equilibrium size. This allows for the deconvolution of temperature induced kinetic changes from changes in kinetics due to possible particle growth with increasing temperature.

The surface analysis of the NiAg₅₀₀ catalyst shows that not only are Ni and Ag physically associated with each other, as in an alloy, but that Ni can also be driven to the surface of the Ag by changing the chemical environment of the catalyst. Surface science experiments show that Ni is highly oxophilic even within Ag (FIG. 2); therefore Ni will be driven to the surface during oxygen-rich epoxidation conditions. The effect of Ni atoms on the Ag surface starts to become evident as we compare the rates of CO₂ and EO formation between various catalysts. FIG. 6 shows Ag, control Ag (synthesized the identical way as AgNi samples but without the addition of Ni) and NiAg₁₅₀ all show similar behavior in that they have higher rates of CO₂ formation than the catalysts with low concentrations of Ni dopant (NiAg₄₀₀ and NiAg₅₀₀). The behavior of the Ag samples is consistent with previously reported values. For example, with the control Ag at 160° C., we measured an EO rate of 0.58 μmols⁻¹g_{Ag}⁻¹, compared to 0.17 μmols⁻¹g_{Ag}⁻¹ on pure Ag catalyst.¹⁵ At a higher temperature of 180° C., an EO rate of 1.4 μmols⁻¹g_{Ag}⁻¹ was measured, compared to a previous report of 2.2 μmols⁻¹g_{Ag}⁻¹.¹⁶ For temperatures higher than 230° C., all the pure Ag catalysts also show a plateauing rate of EO formation at 5.2 μmols⁻¹g_{Ag}⁻¹. Without being bound by a particular scientific theory, this plateauing is likely due to secondary combustion of EO on Ag to form CO₂. NiAg₁₅₀ shows similar behavior to pure Ag, likely because there is a high Ni surface coverage at this relatively “higher” Ni:Ag molar ratio causing extended Ni oxide islands to form, which likely migrate to the Al₂O₃ support. Thus, the behavior of NiAg₁₅₀ does not differ from Ag significantly. For the more dilute NiAg catalysts, NiAg₄₀₀ and NiAg₅₀₀. the data shows the rate of EO production increases from 7 μmols⁻¹g_{Ag}⁻¹ at 200° C. to 45 μmols⁻¹g_{Ag}⁻¹ at 250° C. For NiAg₄₀₀ (made identically as the NiAg₅₀₀ catalyst except with 20% more Ni precursor added during synthesis) and NiAg₅₀₀, the limit of single atom alloys (as seen from FTIR of NiAg₅₀₀) has likely been reached (where Ni stays dispersed, rather than forming contiguous Ni—Ni). This is evident in the significantly higher rates of EO formation seen for these catalysts. In the temperature regime tested, there is also no longer any plateauing of the EO formation rate indicating that the secondary combustion of EO is not present for the doped catalysts. The behavior of the control Ag and its similarity to the Ag catalyst also suggests that the Na (present in NaOH during synthesis) does not affect selectivity significantly in the system.

### (v) Ethylene Epoxidation

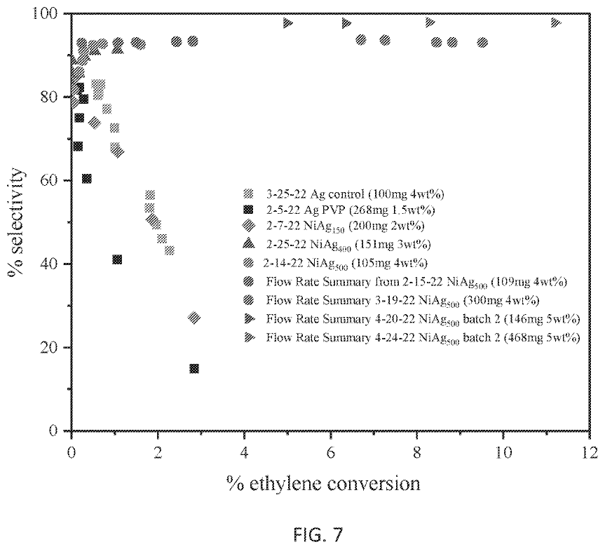

FIG. 7 shows ethylene epoxidation on pure Ag catalysts is characterized by decreasing selectivity with increasing conversion (percentage of ethylene fed to the reactor that is consumed by the catalyst bed)—a trend commonly seen across unpromoted Ag catalysts.^{14.17.18} Higher conversions of ethylene result in an increase in the rate of formation of EO and subsequently the rate of secondary combustion of EO. This is consistent with observations from a study by Keijzer et al.¹⁹ In Keijzer et al.'s work, on 3.75 mg of Ag on 8 m²/g α-Al₂O₃ selectivity drops from 45 to 30% as conversion increases from 1 to 2%. For our control silver, as we increase conversion from 1 to 2%, selectivity decreases from 68 to 46%. Considering that our support has a lower surface area than 8 m²/g implying lower acidity, the observed selectivities on pure Ag catalysts and their dependence on ethylene conversion are consistent with published data. With the NiAg₄₀₀ and NiAg₅₀₀ samples, we see that not only is the selectivity significantly higher than on unpromoted Ag catalysts, but that high selectivity is retained over the conversion range tested. For the NiAg₅₀₀ sample (purple circle), the data shows that a selectivity of 93% is maintained even as ethylene conversion increases from 1 to 2%. These results imply that secondary combustion of EO is likely strongly mitigated on the NiAg surfaces without compromising the rate of ethylene oxide formation. A replicate of NiAg₅₀₀ (NiAg₅₀₀ batch 2 that was synthesized about 2 months after the first batch) showed a selectivity as high as 97% at an ethylene conversion of 11%; at these conditions we measured the outlet EO mol % to be 1.4%. This shows that the very high EO selectivity of NiAg catalysts with dilute Ni loadings, even at increasing ethylene conversion, is reproducible across multiple batches of the material. We expect that with further optimization of the synthesis approach and type of Al₂O₃ used, a higher selectivity at higher conversion could be achieved. We note that ethylene conversion was varied by changing the total gas flow rate, and changing the amount of catalyst in the bed, which both influence the residence time of the gas in the catalyst bed.

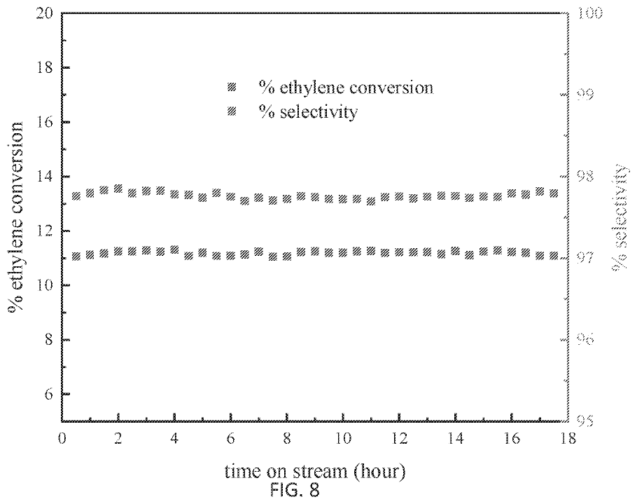

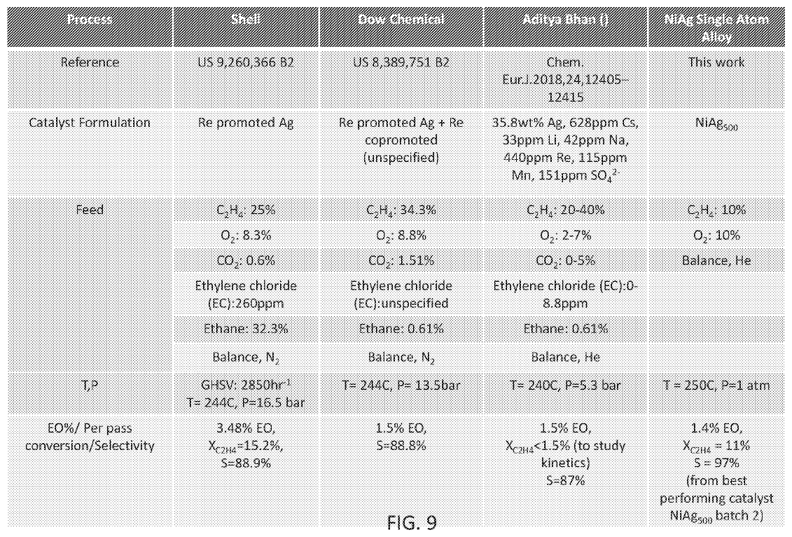

A stability test of the replicate of NiAg₅₀₀ (batch 2) was performed as it operated with the highest selectivity at highest conversion observed thus far (FIG. 8). During the 18-hour run (which was after the catalyst had already been on stream for 2-3 days under various reaction conditions), the rate of conversion and selectivity showed no measurable change. This suggests that the catalyst is quite stable under the reaction conditions tested. In this case a selectivity of ˜98% at an ethylene conversion of ˜11% was observed. Table 2 in FIG. 9 compares this result to the best results we found in the patent and academic literature. Table 2 clearly shows that the NiAg₅₀₀ catalyst outperforms available data from highly optimized catalysts from the primary

EO producers.

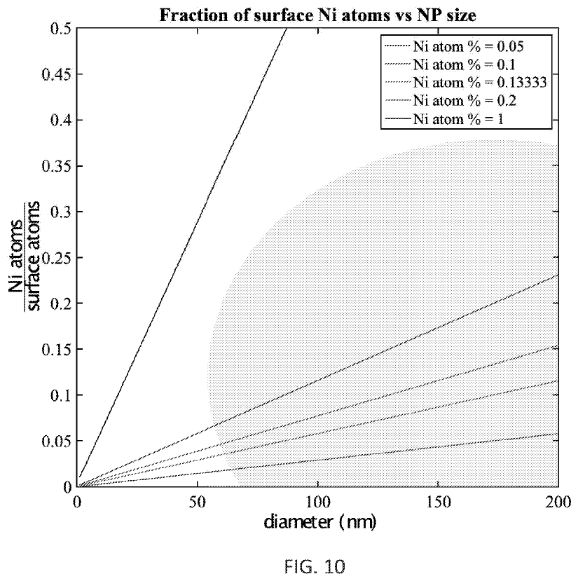

FIG. 10 plots the relationship between surface concentration of Ni atoms and size of the Ag particles for NiAg₅₀₀ , showing that the surface concentration of Ni atoms can be in a range of 8%-20% for the selectivity and ethylene conversion rates described herein. The shaded circle defines example ranges of nanoparticle sizes (e.g., at least 50 nm diameter) and fractions (e.g., Ni atoms/total surface atoms of 35% or less) that are suitable for catalyzing an alkene epoxidation reaction as described herein.

FIG. 10 further illustrates the discovery that a catalyst comprising particles with <dp>=112 and <dp>=120 nm particles (as shown in FIG. 5) and Ni:Ag molar ratio=1:500 surprisingly and unexpectedly increases the selectivity to greater than 85% for the direct epoxidation reaction of ethylene. In another example, using 70 nm average diameter particles and systematically varying Ni loading, a Ni:Ag of 1:200 ratio was observed to maximize selectivity at 77%. Given that smaller particles are often less selective [14], we expect that selectivity increases above 83% using larger particles.

Thus, the data presented herein, including FIG. 7, FIG. 8, and FIG. 10, further supports that 1:100≤Ni:Ag≤1:1000, with particles having an average diameter D (<dp> or <dp>_{SA}) 50 nm≤D≤250 nm (so that 4*10\hat{ }−6≤Ni:Ag/D≤2*10\hat{ }−4 in units of nm\hat{ }−1) also increases the selectivity to greater than 85% for the direct epoxidation reaction of ethylene.

### (v) Method for Defining and Measuring Selectivity of the Epoxidation in FIG. 8

Ultra high purity (UHP) He (Airgas, HE UHP300), C₂H₄ (Airgas, EY UHP300), O₂ (Airgas, OX UHP300), C₂H₄O (Linde, EO 3.0-LB) and H₂ (Airgas, HY UHP300) gases along with 10% CO₂ in Ar (Airgas, AR CD10300) were used during the experiment. The reactants and products were quantified using TCD and FID in a SRI 8610C GC equipped with a methanizer. Calibrations for EO and CO₂ are used to calculate the partial pressures of the products and calculate the molar flow rate of products. C₂H₄ and O₂ are also calibrated to calculate conversions and close mass balances. Selectivity is defined as the following:

$${S}_{EO} = \frac{{F}_{EO}}{{F}_{EO} + 0.5 {F}_{CO 2}}$$

where, F_{E-}, F_{CO2} are molar flow rates of EO and CO₂ respectively and S_{EO} is the selectivity to EO.

### 3. Example Process Steps

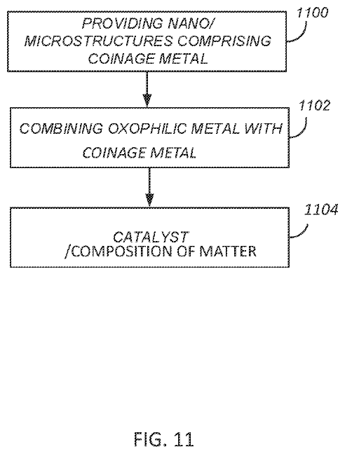

FIG. 11 is a flowchart illustrating a method of making and using a catalyst. The method comprises the following steps.

Block 1100 represents providing/obtaining structures (e.g., nanostructures or microstructures) comprising a coinage metal, e.g., optionally on a substrate or support. Example coinage metals include, but are not limited to, silver, gold, or copper. Example materials for the substrate or support include, but are not limited to, alumina, silica, titania, ceria, zirconia, magnesia, tin oxide, zinc oxide, indium oxide, and zeolite, with surface areas from 0.1-1000 m²/g of the support. The composition of the support may also be tuned to optimize performance of the catalyst.

In one or more examples, the nanostructures or microstructures are further provided with a promoter species for modifying the catalytic activity, selectivity or stability of the composition of matter for the epoxidation reaction. Example promoter species include, but are not limited to, Cs, Re, or Cl or other promoters as known in the art.

In one or more examples, the step comprises providing a colloidal solution comprising the nanostructures or microstructures (other approaches known for depositing metals, metal salts, or metal precursors are also considered). However, the nanostructures or microstructures can take a variety of forms including, but not limited to, particles of any shape (spheres, elongated particles, fibers, nanotubes, etc.), or a nanostructured or micro-structured surface of a film or a porous structure. The nanostructures or microstructures have at least one dimension in a range of 1-10000 nm that can be selected to obtain a desirable atomic dispersion of the oxophilic atoms as described in the next step.

Block 1102 represents forming the structures each combining a coinage metal and an oxophilic metal in a catalytically effective ratio wherein the structures selectively catalyze a direct alkene epoxidation reaction using molecular oxygen (O₂) as an oxidant, when the structures are catalytically activated and used as a catalyst in the epoxidation reaction under reaction conditions.

In one example, the step comprises combining the nanostructures or microstructures with the oxophilic atoms. In one or more examples, the combining may form a plurality of single oxophilic metal atoms atomically dispersed and alloyed into a surface of the structures). Example oxophilic metal atoms include, but are not limited to, nickel, indium, or gallium.

In one or more examples, the combining step comprises combining a reducing agent and an oxophilic metal precursor with the colloidal solution; and performing a sequential reduction reaction wherein oxophilic metal is deposited from the precursor on the nanostructures or microstructures. In one or more examples, the reduction reaction may form a plurality of single oxophilic metal atoms atomically dispersed and alloyed into a surface of the structures.

In one or more examples, an amount of the oxophilic atoms and/or a size of the structures comprising nanoparticles or microparticles are selected to obtain the catalytically effective atomic ratio of the oxophilic metal to the coinage metal. Example stochiometric ratios of the oxophilic metal atoms: coinage metal in each of the structures include, but are not limited to, a stochiometric ratio in a range of 1:3 to 1:10000, 1:3 to 1:1000, 1:10 to 1:1000, or 1:10 to 1:500, for example. Example surface concentrations of the oxophilic metal atoms (number of oxophilic metal atoms/total number of surface atoms)×100 include, but are not limited to, a surface concentration in a range of 0.001%-33%, 0.01% to 20%, 0.1% to 20%, or 1% to 20%, for example. Example dimensions for the structures include, but are not limited to, at least one dimension in a range of 1 to 10000 nanometers, 10 nm to 1000 nm, 1 nm to 500 nm, or 10 nm to 500 nm, wherein the oxophilic atoms are deposited on the nano or micro scaled dimension. In one or more examples, the nanostructures or microstructures comprise particles having a diameter comprising the nanoscale or microscale dimension.

Block 1104 represents the end result, a composition of matter useful as a catalyst in an alkene epoxidation reaction (e.g., as illustrated in FIG. 1).

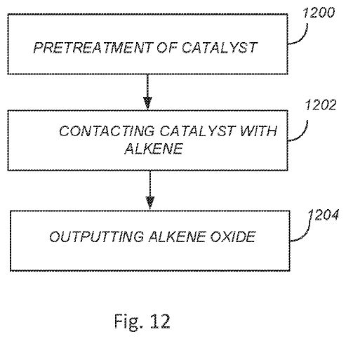

FIG. 12 is a flowchart illustrating a method of using the composition of matter as a catalyst in a reactor for performing an alkene epoxidation reaction, such as an ethylene or propylene epoxidation reaction, comprising the following steps. Block 1200 represents pretreating the catalyst composition (e.g., in H₂); Block 1202 represents contacting the catalyst with an alkene and molecular oxygen; and Block 1204 represents outputting an alkene oxide formed by the reaction.

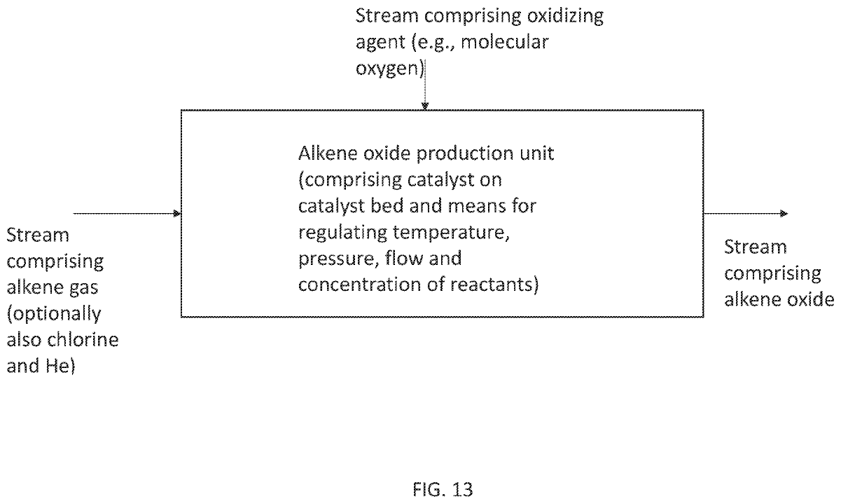

FIG. 13 illustrates an example reactor for performing the epoxidation reaction, wherein temperature and pressure in the reactor, total gas flow rate of the reactants (alkene and molecular oxygen and optionally chlorine and/or a carrier gas such as He), and specific concentrations of the reactants and the catalyst can be selected to optimize the selectivity and alkene conversion. In one or more examples, the catalyst enables alkene epoxidation without the use of chlorine and the reactor does not include a feed for feeding chlorine to the reaction.

In the alkene oxide production step of the present process, alkene from the alkene containing stream is contacted with an oxidizing agent in another stream. The oxidizing agent may be high-purity oxygen or air, but is preferably high-purity molecular oxygen which may have a purity greater than 90%. Typical reaction pressures are 1-40 bar and typical reaction temperatures are 100-400° C. (e.g., 150-250° C.) with concentration of alkene and oxygen in each of their respective streams in a range of 5%-40%, and flow rates of each of the alkene containing stream and the oxygen containing stream in a range of 40-60 sccm. However, temperatures, pressures, reactant concentrations, composition, and flow rates may be those typically used in the art, e.g., as described in U.S. Pat. Nos. 9,260,366 and 8,389,751 which are incorporated by reference herein.

In one or more examples, a concentration of the oxophilic atoms comprising nickel on the surface of Ag nanoparticles increases selectivity to EO to greater than 85%, or greater than 90%, or greater than 95% for the epoxidation reaction CH₂═CH₂+½O₂→(CH₂)₂O, over combustion of ethylene forming carbon dioxide (CH₂═CH₂+3O₂→2CO₂+2H₂O), and for an ethylene conversion of greater than 5%, or greater than 10% or in a range of 5-30%. These values are obtained for the reaction conditions listed in Table 2. As used herein, selectivity of greater than X % means that greater than X % of the ethylene reacts in the reaction CH₂═CH₂+½O₂→(CH₂)₂O and less than (100-X) % of the ethylene reacts in the reaction CH₂═CH₂+3O₂→2CO₂+2H₂O. A conversion greater than Y % means that at least Y % of the alkene inputted into the reactor is converted to alkene oxide or CO₂.

### Example Embodiments

Illustrative embodiments of the inventive subject matter include, but are not limited to, the following (referring also to FIGS. 1-13).
1. A composition of matter 100, comprising:
   particles 102 comprising a first component 104 and a second component 106 in a catalytically effective atomic ratio wherein:
   the particles selectively catalyze a direct alkene epoxidation reaction using molecular oxygen (O₂) as an oxidant, when the particles are catalytically activated and used as a catalyst in the epoxidation reaction under reaction conditions, and the first component comprises at least one of silver, gold, or copper and the second component comprises at least one of nickel, indium, or gallium.
2. A composition of matter 100 useful for catalyzing a direct alkene epoxidation reaction using molecular oxygen (O₂) as an oxidant, comprising:
   a plurality of structures 102 comprising nanostructures or microstructures each comprising a coinage metal 104 and a plurality of single oxophilic metal atoms 106 combined with (e.g., atomically dispersed on, and alloyed into) a surface of the coinage metal, wherein:
   an atomic ratio of oxophilic metal:coinage metal is in a range 1:3≤oxophilic metal:coinage metal≤1:million, and
   the oxophilic metal is characterized by an oxide formation enthalpy metal is characterized by an oxide formation enthalpy (e.g. from a bulk metal consisting of the oxophilic metal atoms) being more exothermic than the coinage metal.
3. The composition of matter of example 1 or 2, wherein the catalytically effective atomic ratio R=first component: second component or oxophilic metal:coinage metal is in a range 1:3≤R≤1: million, or 1:200≤R≤1:1000, or 1:100≤R≤1:1000, or 1:200≤R≤1:500, or 1:100≤R≤1:500.
3. The composition of matter of any of the examples 1-3, wherein the catalytically effective ratio is such that at least 50%, or 5-50%, 10%-50%, 1%-50%, or 25%-35%, or 33% of the surface of each of the NiAg particles is composed of nickel under the reaction conditions.
4. the surface of the AgNi particles are composed of the nickel under the reaction conditions.
5. The composition of matter of any of the examples 1-4, wherein the nickel is oxidized nickel, or the oxophilic metal is oxidized metal, prior to being used as a catalyst and/or prior to preconditioning.
6. The composition of matter of any of the examples 1-5, wherein a majority of the particles or structures each have largest dimension D such that 1 nm≤D≤500 nm, 5 nm≤D≤500 nm, 50 nm≤D≤250 nm, 100 nm≤D≤250 nm, 200 nm≤D≤500 nm, or 1 nm≤D≤10000 nm.
7. The composition of matter of any of the examples 1-6, wherein the particles or structures comprise less than 10 parts per million of any alkali metal element or promoter such as, but not limited to, caesium, lithium, sodium, potassium, or rubidium.
8. The composition of matter of any of the examples 1-7, wherein the catalytically effective atomic ratio is such that the epoxidation reaction proceeds in an absence of chlorine.
9. The composition of matter of any of the examples 1-8, wherein the catalytically effective ratio increases selectivity to greater than 85% or greater than 83% for the direct epoxidation reaction CH₂═CH₂+½O₂→(CH₂)₂O over combustion of ethylene forming carbon dioxide, and wherein greater than 5% of the ethylene is converted to ethylene oxide.
10. The composition of matter of any of the examples 1-9, wherein the catalytically effective atomic ratio R is:
   1:100≤R≤1:1000, the particles have an average diameter D (e.g., <dp> or <dp>SA) 50 nm≤D≤250 nm, and 4*10\hat{ }−6≤Ni:Ag/D≤2*10\hat{ }−4 in units of nm\hat{ }−1, or
   1:100≤R≤1:1000, the particles have an average diameter D is 1 nm≤D≤500 nm, or
   1:200≤R≤1:1000, the particles have an average diameter D is 50 nm≤D≤250 nm,
   1:100≤R≤1:500, the particles have an average diameter D is 1 nm≤D≤500 nm,
   1:200≤R≤1:500, the particles have an average diameter D is 1 nm≤D≤500 nm,
   1:300≤R≤1:500, the particles have an average diameter D is 1 nm≤D≤500 nm,
   1:200≤R≤1:500, the particles have an average diameter D is 50 nm≤D≤250 nm,
   1:300≤R≤1:500, the particles have an average diameter Dis 50 nm≤D≤250 nm,
11. A catalyst for the epoxidation reaction comprising the composition of matter of any of the examples 1-11.
12. The composition of matter of any of the examples including example 2, wherein the coinage metal comprises silver, gold or copper and the oxophilic metal atoms comprise nickel, indium, or gallium.
13. The composition of matter of any of the examples 1-12, wherein the structures or particles further comprise a promoter species for promoting a catalytic activity of the composition of matter for the epoxidation reaction.
14. The composition of matter of any of the examples 1-13, wherein a surface concentration of the oxophilic metal atoms (e.g,. nickel, indium, or gallium) comprising the number of oxophilic metal atoms/total number of surface atoms)×100 is in a range of 0.001%-70%.
15. The composition of matter of any of the examples 1-14 further comprising a support 108 or substrate supporting the structures or particles.
16. The composition of matter of example 15, wherein the support or substrate comprises at least one of aluminum oxide, silica, titania, ceria, zirconia, magnesia, tin oxide, zinc oxide, indium oxide, and zeolite with surface areas from 0.1-1000 m² per gram of catalyst.
17. The composition of matter of any of the examples 2-16, wherein the structures comprise a nanostructured or micro-structured surface 110 of a film or a porous structure.
18. The composition of matter of any of the examples 1-17, wherein the epoxidation reaction comprises alkene epoxidation forming ethylene oxide (ethylene epoxidation) or propylene oxidation.
22. The composition of matter of any of the examples 1-18, wherein the first component or the oxophilic metal is nickel, and the second component or the coinage metal is silver, so that the catalytically effective atomic ratio R is Ni:Ag.
23. The composition of matter of any of the examples 1-19, wherein the oxophilic metal is characterized by at least one of:
   an oxygen adsorption energy O_{A} for adsorbing oxygen on a crystal surface consisting of the oxophilic atoms, such that −5.8 eV≤O_{A}≤−5.4 eV as calculated using density functional theory (DFT) with the PW91 functional, a 396 eV cutoff, a 7×7×1 k-point grid for a 3×3×4 surface cell and according to the method and parameters in [19], and using gas-phase species of O as a reference state,
   a hydroxyl adsorption energy OH_{A} for adsorbing a hydroxyl group on the crystal surface, −3.12 eV≤OH_{A}≤−2.77 eV, as calculated using DFT with the PW91 functional, a 396 eV cutoff and a 7×7×1 k-point grid for a 3×3×4 surface cell according to the method and parameters in [19], and using gas-phase species of OH as a reference state, or
   an O₂ dissociation barrier O_{B} for adsorbing oxygen on a single metal alloy of the oxophilic metal on the coinage metal consisting of Ag, such that 0.00 eV≤O_{B}≤0.25 eV, and a 2O adsorption energy 2O_{A} for adsorbing oxygen on the single metal alloy such that −2.62 eV≤2O_{A}≤−1.50 eV, as calculated using DFT with the PBE functional with TS correction, 400 eV cutoff and a 7×7×1 k-point grid for a 3×3×4 surface cell using the method and parameters in [20], and using adsorbed O₂ as the initial state for calculating O_{B} and gas-phase O₂ was as the reference state for calculating O_{A}.
   metal is characterized by an oxide formation enthalpy from a bulk metal (consisting of the oxophilic metal atoms) being more exothermic than the coinage metal.
21. The composition of matter of any of the examples 1-20, wherein each of the single oxophilic metal atoms is spaced from a next nearest one of the oxophilic atoms by at least one of an oxygen atom, a coinage metal atom, or another type of atom different from the oxophilic metal atom, thereby preventing bonding between the single oxophilic atoms and increasing a number of oxide bonds between the oxophilic atoms and oxygen from the molecular oxygen during the epoxidation.
22. A reactor for performing the epoxidation reaction, comprising an input for receiving the composition of matter of any of the examples 1-21 (e.g., comprising a catalyst or comprising a composition of matter that has been reduced).
23. The reactor of example 22, wherein the reactor does not include a feed for feeding chlorine to the reaction.
24. A method of catalyzing an epoxidation reaction, comprising:
   contacting an epoxidation catalyst with an alkene and molecular oxygen, wherein the epoxidation catalyst comprises a coinage metal and an oxophilic metal, wherein the catalyst selectively catalyzes a reaction comprising a direct epoxidation of the alkene using the molecular oxygen (O₂); and
   outputting an alkene oxide formed by the reaction.
25. The method of example 22, wherein the coinage metal comprises silver and the oxophilic metal comprises nickel.
26. The method of example 24 or 25, further comprising pretreating the oxophilic metal in hydrogen (H2) prior to catalyzing the reaction.
27. The method of any of the examples 24-26, further comprising performing the reaction without feeding chlorine to the reaction.
28. The method of any of the examples 24-27 wherein the epoxidation catalysit comprises the composition of matter of any of the examples 1-21.
29. A method of making a composition of matter, comprising:
   forming structures (e.g., particles) each combining a coinage metal and an oxophilic metal in a catalytically effective ratio wherein the structures selectively catalyze a direct alkene epoxidation reaction using molecular oxygen (O₂) as an oxidant, when the structures are catalytically activated and used as a catalyst in the epoxidation reaction under reaction conditions.
30. The method of example 29, wherein the forming further comprises:
   providing a colloidal solution of the structures comprising nanostructures or microstructures comprising the coinage metal;
   combining a reducing agent and the oxophilic metal precursor with the colloidal solution; and
   performing a sequential reduction reaction wherein oxophilic metal is deposited from the precursor on the nanostructures or microstructures (e.g., so as to form a plurality of single oxophilic metal atoms atomically dispersed and alloyed into a surface of the structures).
31. The method of example 30, wherein a concentration of the precursor and a diameter of the structures (comprising nanoparticles or microparticles) are selected to obtain a catalytically effective atomic ratio of the oxophilic metal to the coinage metal.
32. The method of example 30 or 31, wherein the coinage metal comprises silver, gold or copper and the oxophilic metal comprises nickel, indium, or gallium.
33. The composition of matter of any of the examples 1-21 fabricated using the method of any of the examples 29-32.
34. A composition of matter useful for catalyzing an alkene epoxidation reaction using molecular oxygen (O₂) as an oxidant, comprising:
   a plurality of structures comprising nanostructures or microstructures each comprising a coinage metal; and
   a plurality of single gallium, indium, or nickel atoms attached as a single atom alloy to a surface of each of the structures.
35. The composition of matter of example 34, comprising the composition of matter of any of the examples 1-21.
36. The composition of matter of any of the examples 1-35, wherein the oxophilic atoms (e.g. nickel atoms) do not form oxophilic atom-oxophilic atom (e.g., Ni—Ni) bonds.
37.The composition of matter of any of the examples 1-36, wherein the particles or structures consist of, or consist essentially of the oxophilic metal and the coinage metal.

### 4. Advantages and Improvements

A catalyst that consists of dilute Ni doped in Ag as the only promoter has been shown to enable >90% selectivity at ethylene conversions up to ˜12%, which surpasses the performance of catalysts reported in the literature. We attribute this surprising and unexpected increase in the catalytic performance to the selection of ranges for catalytically effective ratios of the oxophilic metal (Ni) to the coinage metal (silver) and/or sizes of the particles/structures comprising the catalyst.

Further optimization of the catalyst and alkene epoxidation reaction process is expected to result in further improvements. Because catalysts are changed in ethylene oxide producing plants every ˜1-3 years, this catalyst can serve as a “drop in” replacement for current catalyst technologies with minimal lead time to implementation. As a result, existing reactors performing alkene epoxidation reactions using the catalyst described herein would have an almost immediate (the time scale of further optimization and mass production of the catalyst) operational advantage as compared to reactors that do not utilize the catalyst.

### REFERENCES

The following references are incorporated by reference herein
1. Pu, T., Tian, H., Ford, M. E., Rangarajan, S. & Wachs, I. E. Overview of Selective Oxidation of Ethylene to Ethylene Oxide by Ag Catalysts. (2019) doi:10.1021/acscatal.9b03443.
2 Linic, S. & Barteau, M. A. Formation of a stable surface oxametallacycle that produces ethylene oxide. J Am Chem Soc 124, 310-317 (2002).
3 Carbonio, E. A. et al. Are multiple oxygen species selective in ethylene epoxidation on silver? Chemical Science 9, 990-998 (2018).
4. Ozbek, M. O., Onal, I. & van Santen, R. A. Effect of surface and oxygen coverage on ethylene epoxidation. Topics in Catalysis 55, 710-717 (2012).
5. Gao, X. et al. Core-shell gold-nickel nanostructures as highly selective and stable nonenzymatic glucose sensor for fermentation process. Scientific Reports 2020 10:1 10, 1-10 (2020).
6. Trimpalis, A., Giannakakis, G., Cao, S. & Flytzani-Stephanopoulos, M. NiAu single atom alloys for the selective oxidation of methacrolein with methanol to methyl methacrylate. Catalysis Today 355, 804-814 (2020).
7. Zhang, Z., Nenoff, T. M., Huang, J. Y., Berry, D. T. & Provencio, P. P. Room temperature synthesis of thermally immiscible Ag—Ni nanoalloys. Journal of Physical Chemistry C 113, 1155-1159 (2009).
8. Christopher, P. & Linic, S. Shape-and size-specific chemistry of Ag nanostructures in catalytic ethylene epoxidation. ChemCatChem 2, 78-83 (2010).
9 Pu, T., Tian, H., Ford, M. E., Rangarajan, S. & Wachs, I. E. Overview of Selective Oxidation of Ethylene to Ethylene Oxide by Ag Catalysts. ACS Catalysis 10727-10750 (2019) doi:10.1021/acscatal.9b03443.
10. Xu, X. et al. Application of high-spatial-resolution secondary ion mass spectrometry for nanoscale chemical mapping of lithium in an Al—Li alloy. Materials Characterization 181, 111442 (2021).
11. Darby, M. T., Sykes, E. C. H., Michaelides, A. & Stamatakis, M. Carbon Monoxide Poisoning Resistance and Structural Stability of Single Atom Alloys. Topics in Catalysis 2018 61:5 61, 428-438 (2018).
12. Bandara, A. et al. The effect of co-adsorption of on-top CO on the sum-frequency generation signal of bridge CO on the Ni (111) surface. Chemical Physics Letters 290, 261-267 (1998).
13. Shan, J. et al. NiCu single atom alloys catalyze the CH bond activation in the selective non-oxidative ethanol dehydrogenation reaction. Applied Catalysis B: Environmental 226, 534-543 (2018).
14. Egelske, B. T., Xiong, W., Z Zhou, H. & Monnier, J. R. Effects of the method of active site characterization for determining structure-sensitivity in Ag-catalyzed ethylene epoxidation. Journal of Catalysis (2022) doi:10.1016/j.jcat.2022.03.021.
15. Christopher, P., Xin, H. & Linic, S. Visible-light-enhanced catalytic oxidation reactions on plasmonic silver nanostructures. Nature Chemistry 3, 467-472 (2011).
16. van Hoof, A. J. F., Hermans, E. A. R., van Bavel, A. P., Friedrich, H. & Hensen, E. J. M. Structure Sensitivity of Silver-Catalyzed Ethylene Epoxidation. ACS Catalysis 9, 9829-9839 (2019).
17. Li, H., Cao, A. & Nørskov, J. K. Understanding Trends in Ethylene Epoxidation on Group IB Metals. ACS Catalysis 11, 12052-12057 (2021).
18. Keijzer, P. H., van den Reijen, J. E., Keijzer, C. J., de Jong, K. P. & de Jongh, P. E. Influence of atmosphere, interparticle distance and support on the stability of silver on α-alumina for ethylene epoxidation. Journal of Catalysis 405, 534-544 (2022).
19. Kayode, G. O. & Montemore, M. M. Factors controlling oxophilicity and carbophilicity of transition metals and main group metals, Journal of Materials Chemistry A, 9, 22325 (2021).
20. Monasterial, A. P., Hinderks, C. A., Virayavaree, S. & Montemore, M. M. When more is less: Nonmonotonic trends in adsorption on clusters in alloy surfaces, J. Chem. Phys. 153, 111102 (2020).
21. Aho, A., Eränen, K., Lemus-Yegres, L. J., Voss, B., Gabrielsson, A., Salmi, T. & Murzin, D. Y. Ethylene epoxidation over supported silver catalysts-influence of catalyst pretreatment on conversion and selectivity. Journal of Chemical Technology and Biotechnology 93, 1549-1557 (2018).
22. U.S. Pat. No. 8,546,294.
23. U.S. Pat. No. 8,815,769.
24. U.S. Pat. No. 9,458,121.
25. US Patent Publication No. 20200001277.
26. US Patent Publication No. 20200230580.
27. International Patent Publication No. WO/2019/023158.
28. U.S. Pat. No. 7,259,129.
29. U.S. Pat. No. 8,080,677.
30. U.S. Pat. No. 9,339,798.
31. U.S. Pat. No. 10,300,462.
32. https://patents.google.com/patent/CN1400054A/en. Application NO. CN1400054A Preparation method of silver catalyst containing metal nickel

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A composition of matter, comprising:
particles comprising a first component and a second component in a catalytically effective ratio wherein:
the particles selectively catalyze a direct alkene epoxidation reaction using molecular oxygen (O₂) as an oxidant, when the particles are catalytically activated and used as a catalyst in the epoxidation reaction under reaction conditions, and
the first component comprises at least one of silver, gold, or copper and the second component comprises at least one of nickel, indium, or gallium.

2. The composition of matter of claim 1, wherein the first component comprises silver (Ag), the second component comprises nickel (Ni) and the catalytically effective atomic ratio Ni:Ag is in a range 1:100≤Ni:Ag≤1:1000.

3. The composition of matter of claim 1, wherein the catalytically effective ratio is such that 10%-50% or 5%-50% of the surface of each of the particles is composed of nickel under the reaction conditions.

4. The composition of matter of claim 1, wherein the particles comprise less than 10 parts per million of caesium.

5. The composition of matter of claim 1, wherein the catalytically effective ratio is such that the epoxidation reaction proceeds without a presence of chlorine.

6. The composition of matter of claim 1, wherein the catalytically effective ratio increases selectivity to greater than 85% or greater than 83% for the direct epoxidation reaction CH₂═CH₂+½O₂→(CH₂)₂O over combustion of ethylene forming carbon dioxide, and wherein greater than 5% of the ethylene is converted to ethylene oxide.

7. The composition of matter of claim 1, wherein the catalytically effective atomic ratio is 1:100≤Ni:Ag≤1:1000, the particles have an average diameter D
50 nm≤D≤250 nm and 4*10\hat{ }−6≤Ni:Ag/D≤2*10\hat{ }−4 in units of nm\hat{ }−1.

8. The composition of matter of claim 1, wherein a majority of the particles each have largest dimension D such that 1 nm≤D≤500 nm.

9. The composition of matter of claim 1 configured as a catalyst wherein the particles are catalytically activated for the epoxidation reaction.

10. A composition of matter useful for catalyzing a direct alkene epoxidation reaction using molecular oxygen (O₂) as an oxidant, comprising:
a plurality of structures comprising nanostructures or microstructures each comprising a coinage metal and a plurality of oxophilic atoms, wherein:
a ratio of oxophilic metal: coinage metal is in a range 1:100≤oxophilic metal:coinage metal≤1:1000 and wherein a majority of the structures each have largest dimension D such that 1 nm≤D≤500 nm, and
the oxophilic metal is characterized by an oxide formation enthalpy being more exothermic than that of the coinage metal.

11. The composition of matter of claim 10, wherein the structures comprise less than 10 parts per million of caesium.

12. The composition of matter of claim 10, wherein the structures comprise a nanostructured or micro-structured surface of a film or a porous structure.

13. The composition of matter of claim 10, wherein the epoxidation reaction comprises ethylene epoxidation forming ethylene oxide.

14. The composition of matter of claim 10, wherein a concentration of the oxophilic atoms comprising nickel in the coinage metal comprising silver increases selectivity to greater than 85% or greater than 83% for the direct epoxidation reaction CH₂═CH₂+½O₂→(CH₂)₂O over combustion of ethylene forming carbon dioxide, and wherein greater than 5% of the ethylene is converted to ethylene oxide.

15. The composition of matter of claim 10, wherein the oxophilic metal is characterized by at least one of:
an oxygen adsorption energy O_{A} for adsorbing oxygen on a crystal surface consisting of the oxophilic atoms, such that −5.8 eV≤O_{A}≤−5.4 eV as calculated using density functional theory (DFT) with the PW91 functional, a 396 eV cutoff, a 7×7×1 k-point grid for a 3×3×4 surface cell and according to the method and parameters in [19], and using gas-phase species of O as a reference state,
a hydroxyl adsorption energy OH_{A} for adsorbing a hydroxyl group on the crystal surface, −3.12 eV≤OH_{A}≤−2.77 eV, as calculated using DFT with the PW91 functional, a 396 eV cutoff and a 7×7×1 k-point grid for a 3×3×4 surface cell according to the method and parameters in [19], and using gas-phase species of OH as a reference state, or
an O₂ dissociation barrier O_{B} for adsorbing oxygen on a single metal alloy of the oxophilic metal on the coinage metal consisting of Ag, such that 0.00 eV≤O_{B}≤0.25 eV, and a 2O adsorption energy 2O_{A} for adsorbing oxygen on the single metal alloy such that −2.62 eV≤2O_{A}≤−1.50 eV, as calculated using DFT with the PBE functional with TS correction, 400 eV cutoff and a 7×7×1 k-point grid for a 3×3×4 surface cell using the method and parameters in [20], and using adsorbed O₂ as the initial state for calculating O_{B} and gas-phase O₂ was as the reference state for calculating O_{A}.

16. A reactor for performing the epoxidation reaction, comprising an input for receiving the composition of matter of claim 1 configured as a catalyst.

17. The reactor of claim 16, wherein the reactor does not include a feed for feeding chlorine to the reaction.

18. A method of catalyzing an epoxidation reaction, comprising:
contacting an epoxidation catalyst with an alkene and molecular oxygen, wherein the epoxidation catalyst comprises a coinage metal and an oxophilic metal, wherein the catalyst selectively catalyzes a reaction comprising a direct epoxidation of the alkene using the molecular oxygen (O₂); and
outputting an alkene oxide formed by the reaction.

19. The method of claim 18, wherein the coinage metal comprises silver and the oxophilic metal comprises nickel.

20. The method of claim 18, further comprising pretreating the oxophilic metal in hydrogen (H₂) prior to catalyzing the reaction.

21. The method of claim 18, further comprising performing the reaction in an absence of chlorine.

22. (canceled)

23. (canceled)

23. (canceled)

24. (canceled)
